(19) United States
(12) Patent Application Publication
(10) Pub. No.: US 2015/0204869 A1
(43) Pub. Date: Jul. 23, 2015

(54) Lipocalin-2 as a Biomarker for Pneumococcal Infection Status and Uses Thereof

(71) Applicant: Isis Innovation Limited, Oxford (GB)

(72) Inventors: Climent Casals-Pascual, Oxford (GB); Stephen Howie, Fajara (GM)

(73) Assignee: Isis Innovation Limited, Oxford (GB)

(21) Appl. No.: 14/411,296

(22) PCT Filed: Jul. 3, 2013
(86) PCT No.: PCT/GB2013/051768

§ 371 Date: Dec. 24, 2014

(30) Foreign Application Priority Data
Jul. 5, 2012 (GB) 1211982.2

(51) Int. Cl. G01N 33/569; A61K 45/06

(52) U.S. Cl. CPC G01N 33/56944; G01N 33/56905; A61K 45/06; G01N 2333/4737; G01N 2333/4704; G01N 2333/4703

(57) ABSTRACT

A method for determining the pneumococcal infection status of a subject, comprising the steps of: i, providing a biological sample obtained from a subject: ii. determining the level of lipocalin-2 in the biological sample; and iii. comparing the level determined in (ii) with one or more pre-determined reference values.

The present invention relates to one or more novel biomarkers for determining pneumococcal infection in a subject, and to uses of the one or more biomarkers. The invention may also relate to a means to distinguish between a pneumococcal infection and a malarial infection.

Acute respiratory infections are a leading cause of mortality in children globally. In the developing world pneumonia accounts for 2 million deaths every year in children younger than five years of age. Therefore, there is a continuing need to reduce the number of deaths caused by pneumonia through the implementation of effective preventative measures and improved clinical management.

The diagnosis of pneumonia is usually compromised by the lack of specificity of respiratory symptoms, which are commonly shared with other conditions that cause respiratory distress in children, many of which are associated with very high case fatality rates. In sub-Saharan Africa Plasmodium falciparum malaria and bacterial bloodstream infections are commonly associated with respiratory distress in children and possibly pathogenically linked.

Respiratory distress is the main cause of death in developing countries. Approximately 2.2 million deaths are associated with pneumonia and 780,000 with severe malaria. Most deaths due to severe malaria occur within the first 24 hours of admission, and respiratory distress is a common presentation of severe malaria. Therefore, there is also a continuing need to reduce the number of deaths caused by severe malaria through the implementation of effective preventative measures and improved clinical management and to provide a rapid diagnostic tool which distinguishes severe pneumonia from severe malaria, since critically, the clinical management of these two conditions is different.

Previous studies have shown that delayed referral is one of the most important risk factors for death in children with pneumonia. To ensure that the majority of children with pneumonia receive prompt, adequate treatment, the clinical criteria used to identify and refer patients with acute lower respiratory infection (ALRI) to hospital are commonly based on diagnostic sensitivity rather than specificity. However, in resource-limited areas of the developing world, a clinical definition with low specificity becomes impractical. For a biomarker to be of practical use in such clinical settings, its diagnostic performance has to be superior to current standard methods and its specificity must be sufficient to guide clinical management.

In England and Wales, invasive pneumococcal disease is a leading cause of morbidity and mortality of children causing septicaemia, meningitis and pneumonia. Surveillance of pneumococcal disease in England and Wales is based on the identification of invasive infections, primarily through culture of Streptococcus pneumoniae from normally sterile sites in clinically suspected cases. Current diagnostic techniques therefore require a period of at least 24 hours for bacterial culture confirmation. Moreover, cultures are likely to be negative in those patients that have received antibiotics (false negative results). Therefore, there is a need to develop a rapid test to enable quick diagnosis of pneumococcal infection and one that does not become invalidated in patients that have been previously treated with antibiotics.

It is an aim of the present invention to provide one or more biomarker(s) that may be used to give an indication of pneumococcal infection in an individual and/or which may also be used to assess the type of treatment that is appropriate for such an individual.

In a first aspect, the present invention provides a method for determining the pneumococcal infection status of a subject, comprising the steps of:
i. providing a biological sample obtained from a subject;
ii. determining the level of lipocalin-2 in the biological sample; and
iii. comparing the level determined in (ii) with one or more pre-determined reference values.

The step of obtaining the sample preferably does not form part of the invention,

The term ‘biological sample’ refers to a sample of biological fluid obtained for the purpose of diagnosis or evaluation of a subject of interest. Preferred biological samples include, but are not limited to, blood, serum, plasma, saliva, sputum and cerebrospinal fluid. In addition, the person skilled in the art would realise that some test samples would be more readily analyzed following a fractionation or purification procedure, for example, separation of whole blood into serum or plasma components.

The biological sample may be a plasma sample obtained from a subject.

The term ‘level’ as defined herein refers to the amount or concentration of protein biomarker contained in the biological sample.

The invention may additionally comprise determining the level of one or more of C-reactive protein and von Willebrand factor (vWF), in a biological sample from a subject and comparing the level with one or more pre-determined reference values.

Lipocalin-2 (lpc2), also known as neutrophil gelatinase-associated lipocalin (NGAL), is a 25 kDa secretory glycoprotein that was originally identified in mouse kidney cells and human neutrophil granules. It belongs to the lipocalin superfamily that consists of over 20 small secretory proteins, including RBP4, fatty acid binding proteins (FABP), major urinary proteins (MIR), apolipoprotein D (apoD) and prostaglandin D synthases (PGDS). The common feature of this protein family is their capacity to bind and transport small lipophilic substances, such as free fatty acids, retinoids, arachidonic acid, and various steroids.

C-Reactive Protein (CRP) is an acute-phase inflammatory protein that increases non-specifically in response to tissue injury or infections. CRP is secreted by the liver, principally in response to interleukin-6. CRP is a member of the pentraxin family of proteins and may increase up to 50,000-fold during acute inflammation.

Von Willebrand factor (vWF) is a large multimeric blood glycoprotein (up to 20 000 kDa) involved in haemostasis. It is constitutively produced by endothelial cells, megakaryocytic cells and sub-endothelial connective tissue. The main role of vWF is to mediate platelet adhesion and to bind coagulation factors. vWF is biologically degraded by the metalloproteinase ADAMTS-13. The genetic deficiency of vWF is the most common inherited human bleeding disorder. Besides its role in haemostasis, vWF has also been shown to support inflammatory processes by enhancing leukocyte tethering, rolling and extravasation from blood.

The phrase “pneumococcal infection status” includes any manifestation of pneumococcal infection. For example, the presence or absence of pneumococcal infection, the severity of pneumococcal infection, the progression of pneumococcal infection, and the effectiveness or response of a subject to a treatment for pneumococcal infection. In particular, a concentration of lipocalin-2 greater than about 112 ng/mL, preferably greater than about 118 ng/mL is indicative of a severe pneumococcal infection/severe pneumonia. If this is accompanied by levels of C-reactive protein greater than about 157 mg/mL and/or vWF greater than about 1,915 mU/mL in a biological sample the likelihood of pneumococcal infection/pneumonia in patients increases.

The method of the invention may further include the step of determining if the level of lipocalin-2 is greater than about 118 ng/mL and administering appropriate treatment for a pneumococcal/pneumonia infection if the level of lipocalin-2 is greater than about 118 ng/mL.

The method of the invention may be used, for example, for any one or more of the following: to diagnose pneumococcal infection, in particular, severe or very severe pneumococcal infection; to diagnose pneumonia, in particular, severe pneumonia or very severe pneumonia; to advise on the prognosis of a subject with pneumococcal infection; to monitor disease progression; and to monitor effectiveness or response of a subject to a particular treatment.

Non-severe pneumonia (mild pneumonia) may be defined by the presence of cough or difficulty breathing plus tachypnoea and no signs of severity. The definition of tachypnoea is based on respiratory rate and age (≧50 breaths per minute in children 2-12 months old and ≧40 breaths per minute in children >1.2 months and adults).

Severe pneumonia/severe pneumococcal infection may be defined by the presence of cough or difficulty breathing plus respiratory distress (lower chest wall indrawing or nasal flaring) and/or a positive blood culture or by the presence of consolidation in the chest X-ray.

Very severe pneumonia/very severe pneumococcal infection may be defined as severe pneumonia with oxygen saturation less than 90%.

Preferably the method of the invention allows the diagnosis of pneumonia/pneumococcal infection from the analysis of the level of a biomarker in a sample from the patient.

An elevated level of lipocalin-2, C-reactive protein and/or vWF may indicate the occurrence of severe/very severe pneumonia and/or severe/very severe pneumococcal infection. An elevated level of C-reactive protein may indicate the occurrence of severe pneumonia and/or severe pneumococcal infection. In particular, a concentration of lipocalin-2 greater than about 118 ng/mL is indicative of a severe pneumococcal infection/severe pneumonia. If this is accompanied by levels of C-reactive protein of greater than about 157 μg/mL and/or vWF greater than about 1,915 mU/mL in a biological sample the likelihood of pneumococcal infection/pneumonia in patients increases. Preferably the concentration of lipocalin-2 and CRP and/or vWF is used to diagnose the pneumococcal/pneumonia status of a subject.

A concentration of lipocalin-2 lower than about 79 ng/mL is indicative of non-severe pneumonia. A concentration of CRP lower than about 21 μg/mL is indicative of non-severe pneumonia. A concentration of vWF lower than about 1,082 mU/mL is indicative of non-severe pneumonia.

According to another aspect, the present invention provides a method for determining the pneumococcal infection status or malarial infection status of a subject, comprising the steps of:
i. providing a biological sample obtained from a subject;
ii. determining the level of lipocalin-2 and haptoglobin in the biological sample; and
iii. comparing the level determined in (ii) with one or more pre-determined reference values.

Preferably, the method allows severe pneumonia/pneumococcal infection to be distinguished from malarial infection.

Haptoglobin is a highly abundant plasma protein, which binds the globin chain of free hemoglobin in the blood. The half-life of haptoglobin is approximately five days, but in the presence of free hemoglobin (e.g., malaria-associated intravascular haemolysis), the hemoglobin-haptoglobin complex is rapidly cleared from the plasma by the monocyte-macrophage system through the CD163 receptor with resultant plasma haptoglobin levels that are low or absent.

The phrase ‘malarial infection status’ includes any manifestation of malarial infection. For example, the presence or absence of malarial infection, the severity of malarial infection, the progression of malarial infection, and the effectiveness or response of a subject to a treatment for malarial infection. In particular, a concentration of lipocalin-2 of greater than about about 98.64 ng/mL will be indicative of acute respiratory distress and a haptoglobin concentration of greater than about 666,120 ng/mL in a biological sample may be indicative of pneumonia as the cause of respiratory distress.

A concentration of lipocalin-2 of greater than about 98.64 ng/mL will be indicative of respiratory distress and a concentration of haptoglobin lower than about 627,000 ng/ml is indicative of malaria as the cause of respiratory distress. This may indicate that the patient is 1,925 times more likely to have severe malaria than severe pneumonia (Odds ratio: 1,925, 95% Confidence interval 409-9046).

The method of the invention may be used, for example, for any one or more of the following: to diagnose malarial infection, in particular, severe malarial infection; to diagnose malaria, in particular, severe malaria; to advise on the prognosis of a subject with malarial infection; to monitor disease progression; to distinguish malarial infection from pneumococcal infection and to monitor effectiveness or response of a subject to a particular treatment.

Preferably the method of the invention allows the diagnosis of malaria/malarial infection from the analysis of the level of a biomarker in a sample from the patient.

The reference value against which the level of expression is evaluated may be the level of expression of the same protein in a sample from one or more subjects who do not have pneumococcal infection as determined, for example, by microbiological culture analysis. These samples have so called “normal values” of the biomarkers.

The reference value against which the level of expression is evaluated may be the level of expression of the same protein in a sample from one or more subjects who do not have a malarial infection—as determined, for example, by microbiological culture analysis. These samples have so called “normal values” of the biomarkers.

Alternatively, the reference value may be a previous value obtained for a specific subject. This kind of reference may be used if the method is to be used to monitor progression of an infection or to monitor response of a subject to a particular treatment.

The level of lipocalin-2, C-reactive protein, vWF or haptoglobin may be evaluated by any suitable method. For example if protein levels are to be determined any of the group comprising immunoassays, spectrometry, western blot, ELISA, immunoprecipitation, slot or dot blot assay, isoelectric focussing, SDS-PAGE and antibody microarray immunohistological staining, radio immuno assay (RIA), fluoroimmunoassay, an immunoassay using an avidin-biotin or streptoavidin-biotin system, etc and combinations thereof may be used. These methods are well known to persons skilled in the art. Other methods may also be used.

Preferably the method of the invention allows a result to be obtained in less than 24 hours, preferably less than 12 hours, less 6 hours, less than 4 hours, less than 3 hours, less than 2 hours, less than 1 hour, more preferably less than 30 minutes.

The method of the invention may be used in conjunction with an assessment of clinical symptoms to provide a more effective diagnosis of pneumococcal infection.

The present invention may also provide a method for determining the appropriate treatment for a subject comprising the steps of:
(a) providing a biological sample obtained from a subject;
(b) determining the level of lipocalin-2 in the biological sample from said subject;
(c) comparing the level of lipocalin-2 determined in step 1) with one or more predetermined reference values; and
(d) using the results in (c) to determine the most appropriate therapy.

For example, a patient with clinical signs of pneumonia with a concentration of lipocalin-2 greater than about 118 ng/mL, and optionally also a concentration of CRP greater than about 157 μg/mL and/or a concentration of vWF greater than about 1,915 mU/mL should be referred immediately to hospital for injectable antibiotics and oxygen if needed (in the case of very severe pneumonia), whereas those cases with values below the cut-off values stated above appropriate antibiotics can be prescribed and advise provided to the mother/carer on other supportive measures and when to return for a follow-up visit.

The method of the invention which uses lipocalin-2, and optionally also C-reactive protein and/or vWF, to determine diagnosis of severe/very severe pneumonia and/or severe/very severe pneumococcal infection may be used alone or in combination with one or more known assessment means to determine diagnosis of severe/very severe pneumonia and/or severe/very severe pneumococcal infection.

The known assessment means may include one or more clinical features selected from the group consisting of respiratory rate, the presence of crepitations, crackles on auscultation, respiratory grunting, heart rate, low percentage oxygen saturation (less than 95%), inability to feed and pallor. In a preferred embodiment, the clinical features may be respiratory rate, crackles and inability to feed.

In another embodiment of the invention, the method of the invention which uses lipocalin-2, and optionally also C-reactive protein and/or vWF, to determine a diagnosis of severe/very severe pneumonia and/or severe/very severe pneumococcal infection may be used in combination with the clinical features of respiratory rate, crackles and inability to feed to determine diagnosis of severe/very severe pneumonia and/or severe/very severe pneumococcal infection.

The method of the present invention may be carried out in vitro.

The subject may be a human. In another embodiment the subject may be a mammal, for example, a dog, cat, horse, cow, monkey, ape, rodent, hamster, rat, or guinea pig.

The present invention provides a method of determining the severity of a pneumococcal infection or the response of a pneumococcal infection to a particular treatment in a subject comprising the steps of:
(a) providing a biological sample obtained from a subject;
(b) determining the level of lipocalin-2 in the biological sample from said subject; and
(c) comparing the level of lipocalin-2 determined in step (b) with one or more reference values.

In addition to lipocalin-2, the levels of CRP and/or vWF may also be measured and compared to reference values.

In another aspect, the present invention provides a method of determining the severity of a malarial infection or pneumococcal infection or the response of a malarial infection or a pneumococcal infection to a particular treatment in a subject comprising the steps of:
(a) providing a biological sample obtained from a subject;
(b) determining the level of lipocalin-2 and haptoglobin in the biological sample from said subject; and
(c) comparing the level of lipocalin-2 and haptoglobin determined in step (b) with one or more reference values.

According to yet another aspect of the invention, the invention provides a kit for use in determining the diagnosis of severe/very severe pneumonia and/or severe/very severe pneumococcal infection in a subject comprising at least one agent for determining the level of lipocalin-2, in a biological sample provided by the subject. The kit may further comprise one or more agents for determining the level of one or more of C-reactive protein and/or vWF.

According to yet another aspect of the invention, the invention provides a kit for use in determining the diagnosis of severe malaria or severe pneumococcal infection/severe pneumonia in a subject comprising at least one agent for determining the level of lipocalin-2 and haptoglobin in a biological sample provided by the subject.

The kit may provide an indication useful in determining whether the subject should be referred to hospital due to the severity of the pneumococcal infection or malarial infection.

The agent may be an antibody.

The kit may further comprise instructions for suitable operational parameters in the form of a label or separate insert.

The kit may further comprise one or more lipocalin-2, C-reactive protein and/or vWF samples to be used as standard(s) for calibration and comparison.

The kit may further comprise one or more lipocalin and haptoglobin samples to be used as standard(s) for calibration and comparison.

The invention may further provide use of the level of lipocalin-2, and optionally also one or more of C-reactive protein, vWF and/or haptoglobin, as a biomarker to determine the pneumococcal infection status of a subject.

The invention may further provide use of the level of lipocalin-2 and haptoglobin as a biomarker to determine the pneumococcal infection or malarial status of a subject.

The present invention may provide use of the level of lipocalin-2, C-reactive protein and/or vWF as a means of assessing the most appropriate therapy for an individual with a pneumococcal infection.

The present invention may provide use of the level of lipocalin-2 and haptoglobin as a means of assessing the most appropriate therapy for an individual with a pneumococcal infection or malarial infection.

According to a further aspect the invention provides a method for diagnosing pneumococcal infection in a patient comprising analysing a patient sample to determine the level of lipocalin-2, wherein pneumococcal infection is diagnosed if lipocalin-2 levels are elevated compared with one or more pre-determined reference values.

Preferably if the level of lipocalin-2 in the sample is greater than about 118 ng/mL this is diagnostic of pneumococcal/pneumonia infection.

In a yet further aspect the invention provides a method for diagnosing pneumococcal infection in a patient by analysing a sample from the patient to determine the level of lipocalin-2, and the level of one or more of C-reactive protein and von Willebrand factor (vWF), wherein pneumococcal infection is diagnosed if the level of lipocalin-2, and the level of one or more of C-reactive protein and von Willebrand factor is elevated compared with one or more pre-determined reference values.

Preferably, if the level of lipocalin-2 in the sample is greater than about 118 ng/mL, and the level of C-reactive protein is greater than about 157 mg/mL and/or the level of vWF is greater than about 1,915 mU/mL this is diagnostic of pneumococcal infection/pneumonia.

According to a further aspect the invention provides a method for diagnosing and treating pneumococcal infection in a patient comprising: analysing a patient sample to determine the level of lipocalin-2, wherein pneumococcal infection is diagnosed if lipocalin-2 levels are elevated compared with one or more pre-determined reference values; and administering antibiotics or another therapy for pneumococcal infection to a patient diagnosed with pneumococcal infection.

Preferably if the level of lipocalin-2 in the sample is greater than about 118 ng/mL this is diagnostic of pneumococcal/pneumonia infection.

The method of the invention may further comprise analysing a patient sample to determine the level one or more of C-reactive protein and von Willebrand factor (vWF), wherein pneumococcal infection is diagnosed if the level of lipocalin-2, and the level of one or more of C-reactive protein and von Willebrand factor is elevated compared with one or more pre-determined reference values.

Preferably, if the level of lipocalin-2 in the sample is greater than about 118 ng/mL, and the level of C-reactive protein is greater than about 157 mg/mL and/or the level of vWF is greater than about 1,915 mU/mL this is diagnostic of pneumococcal infection/pneumonia.

According to a still further aspect the invention provides a method for treating pneumococcal infection in a patient comprising: requesting a test to determine whether a patient has elevated levels of lipocalin-2 and administering antibiotics or another therapy for pneumococcal infection to a patient with levels of lipocalin-2 that are elevated compared to a reference value.

According to a yet further aspect the invention provides a method for diagnosing pneumococcal infection in a subject, wherein the pneumococcal infection is characterised by the presence of elevated levels of lipocalin-2 comprising:
i) providing a biological sample obtained from the subject;
ii) applying an antibody, preferably a monoclonal antibody, specific for lipocalin-2 to the sample, wherein presence of lipocalin-2 creates an antibody-lipocalin-2 complex;
iii) applying a detection agent that detects the antibody-lipocalin-2 complex; and
iv) diagnosing pneumococcal infection where an increase in the detection agents of step iii) is detected compared to a reference value.

Preferably if the level of antibody-lipocalin-2 in the sample is greater than seen with a concentration of lipocalin-2 of about 118 ng/mL, then this is diagnostic of pneumococcal/pneumonia infection.

The method may further comprise applying one or more antibodies specific for the C-reactive protein and/or von Willebrand factor (vWF).

According to a further aspect the invention provides a method for diagnosing malaria infection in a patient comprising analysing a patient sample to determine the level of lipocalin-2 and haptoglobin, wherein malaria infection is diagnosed if lipocalin-2 levels are greater than about 98.64 ng/mL and haptoglobin levels are lower than 627,000 ng/mL.

In a further aspect the invention provides a method for diagnosing pneumococcal infection in a patient comprising analysing a patient sample to determine the level of lipocalin-2 and haptoglobin, wherein pneumococcal infection is diagnosed if lipocalin-2 levels are greater than about 98.64 ng/mL and haptoglobin levels are greater than 666,120 ng/mL.

The skilled man will appreciate that preferred features of any one embodiment and/or aspect of the invention may be applied to all other embodiments and/or aspects of the invention.

There now follows by way of example only a detailed description of the present invention with reference to the accompanying drawings, in which:

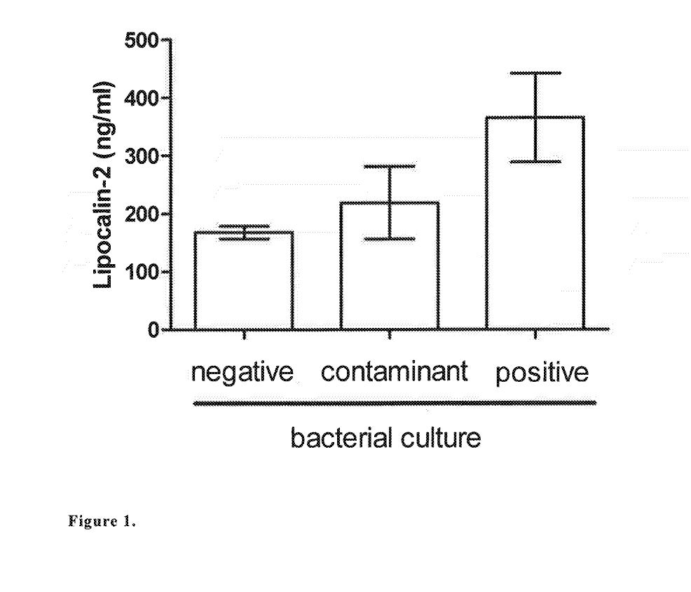

FIG. 1—shows concentration of Lipocalin-2 in plasma samples according to blood culture results from Gambian children in whom blood cultures were performed.

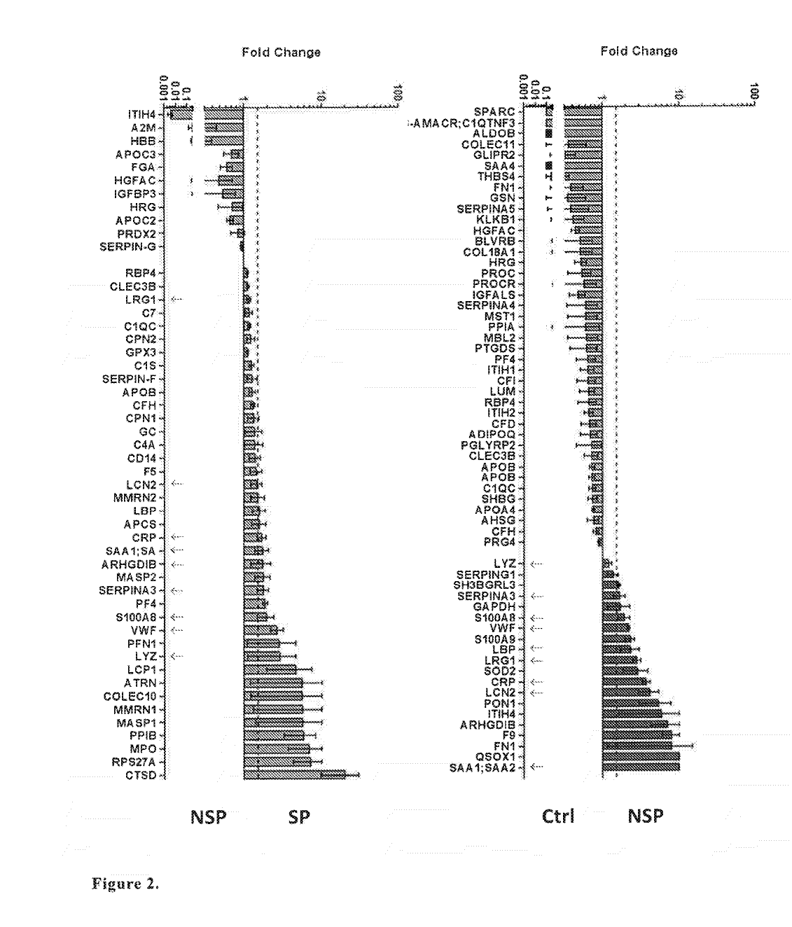

FIG. 2—shows relative abundance of plasma proteins identified and quantified by liquid chromatography tandem-mass spectrometry in children with severe pneumonia (SP), non-severe pneumonia (NSP) and controls (Ctrl). Protein abundance was quantified using normalised spectral counts (SINQ). Error bars indicate standard error of the mean of three independent pools of samples. Red and green bars denote up- and -down-regulation, respectively. Arrows indicate candidate markers of disease progression

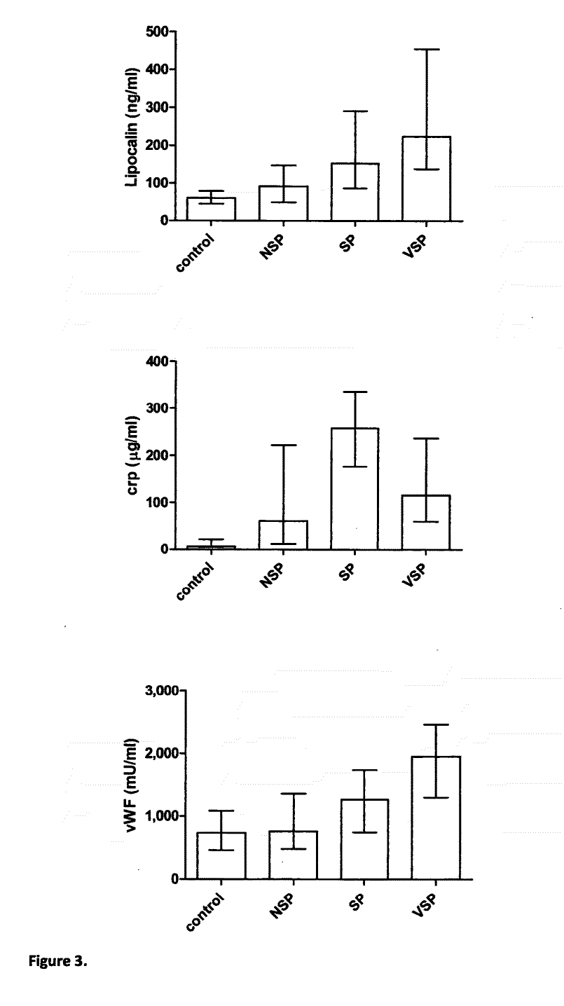

FIG. 3—shows validation of candidate biomarkers in individual clinical samples by ELISA in clinical groups with different severity. Bars indicate median concentration and error bars denote interquartile range (percentile 25 to percentile 75).

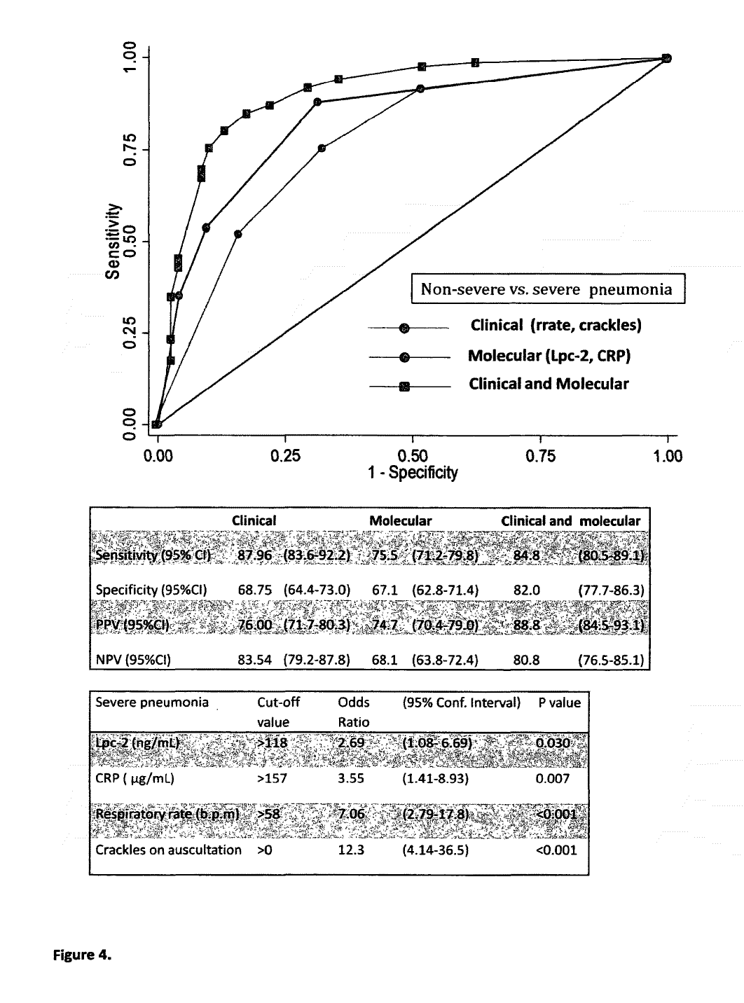

FIG. 4—shows the diagnostic performance of biomarkers in Gambian patients with non-severe pneumonia versus severe pneumonia.

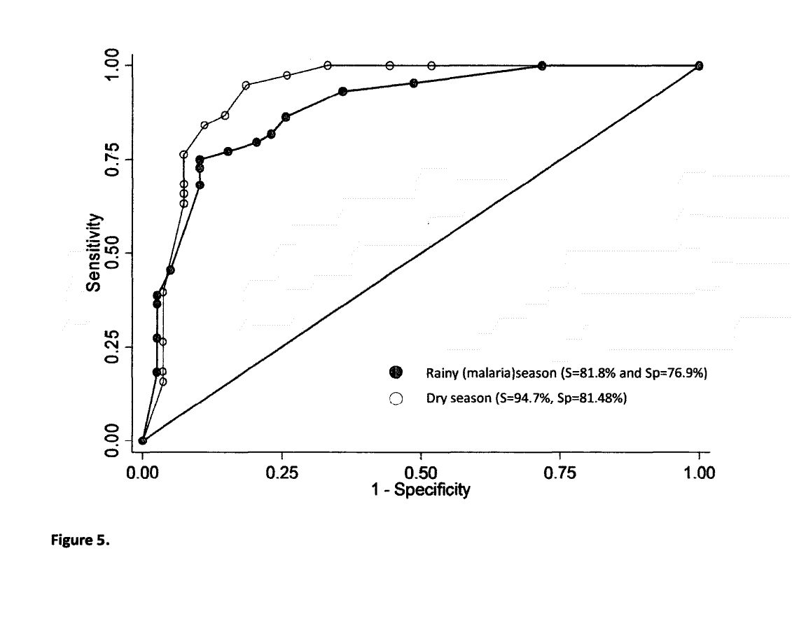

FIG. 5—shows the diagnostic performance of the best model to predict severe pneumonia stratified by seasonality, ROC curves show differences in sensitivity and specificity of the combination of clinical features (respiratory rate and crepitations) and molecular markers (Lpc-2 and CRP) to predict severe pneumonia (versus mild pneumonia). The analysis shows ROC analysis stratified by season of enrolment in the study, 65 patients enrolled in the dry season versus 85 patients in the rainy season. The proportion of severe and mild pneumonia cases did not vary significantly with season (chi-square: 0.46). Black circles denote rainy/malaria season (June to November) and empty circles denote dry season.

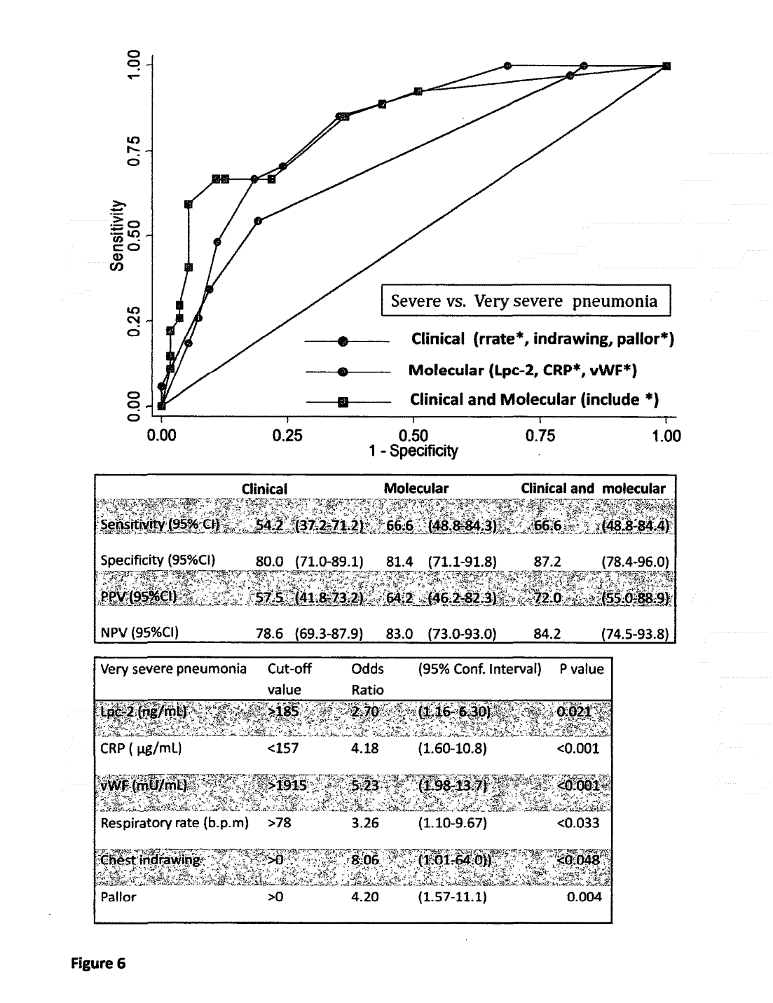

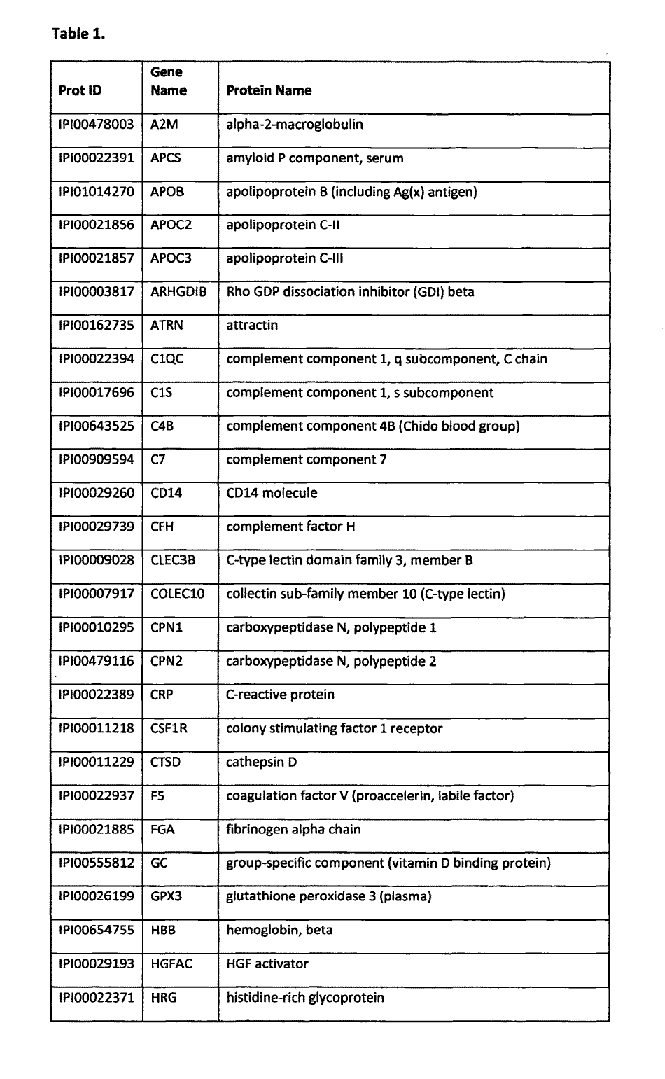

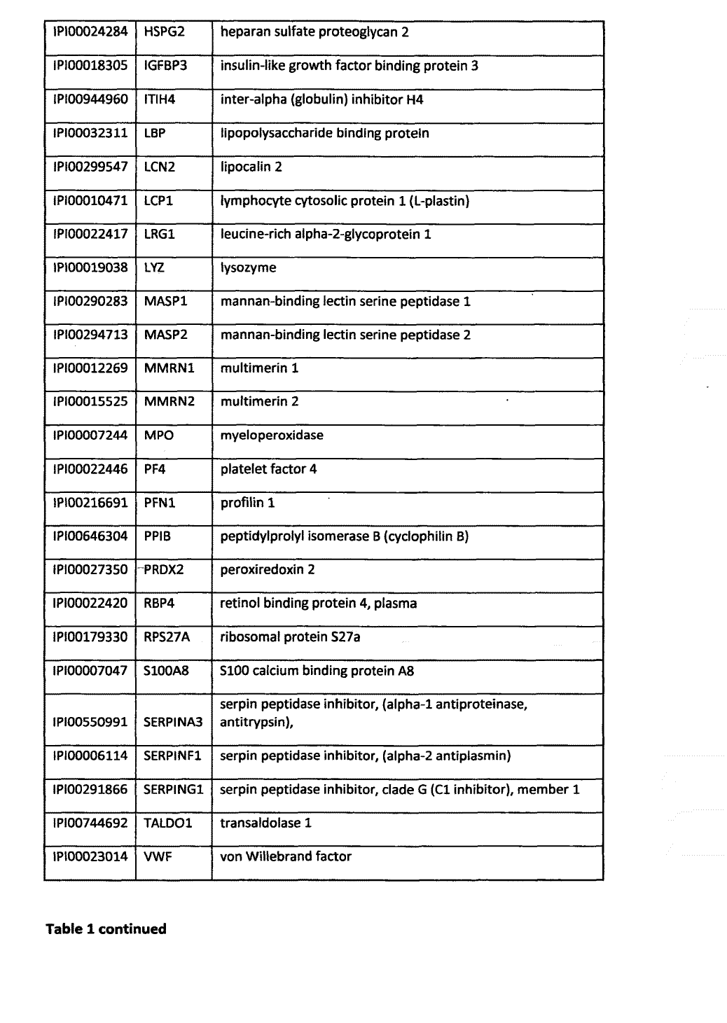

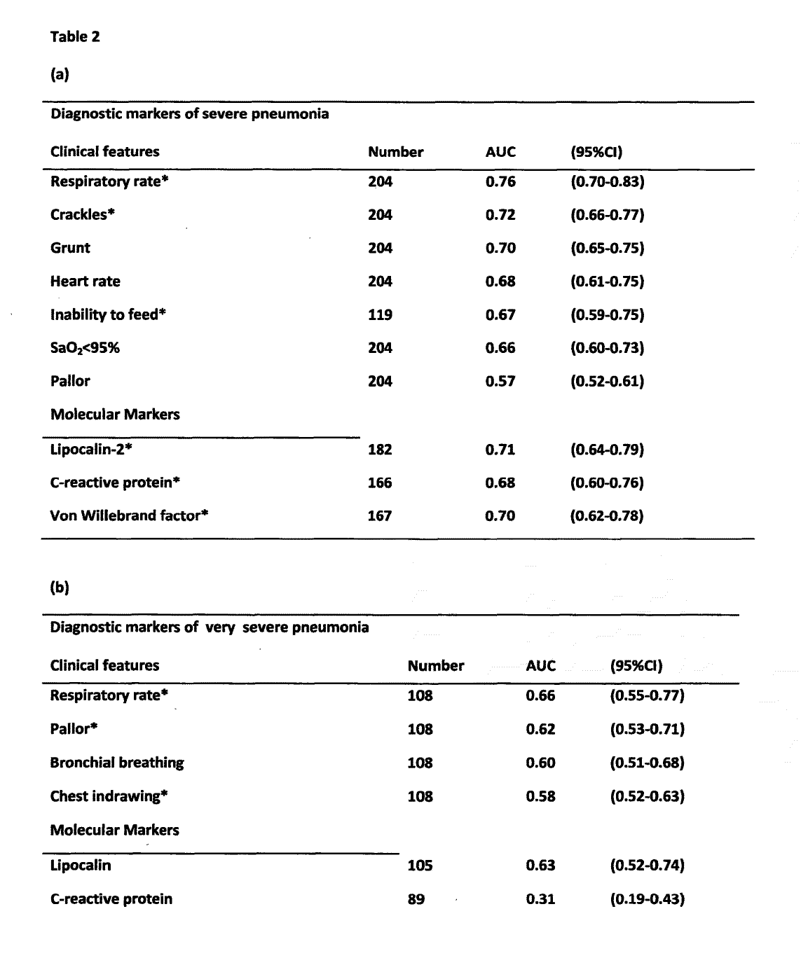

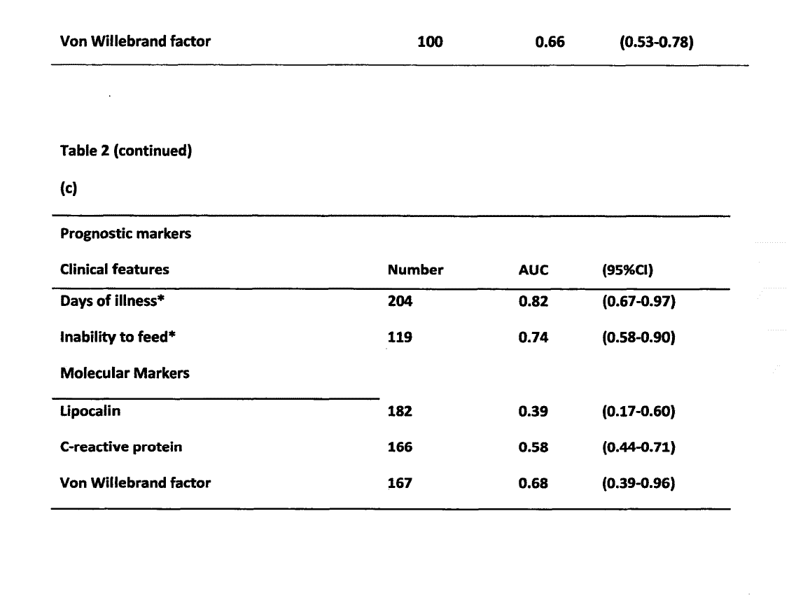

FIG. 6—shows the diagnostic performance of biomarkers in Gambian patients with severe versus very severe pneumonia (saturation of oxygen<90%).

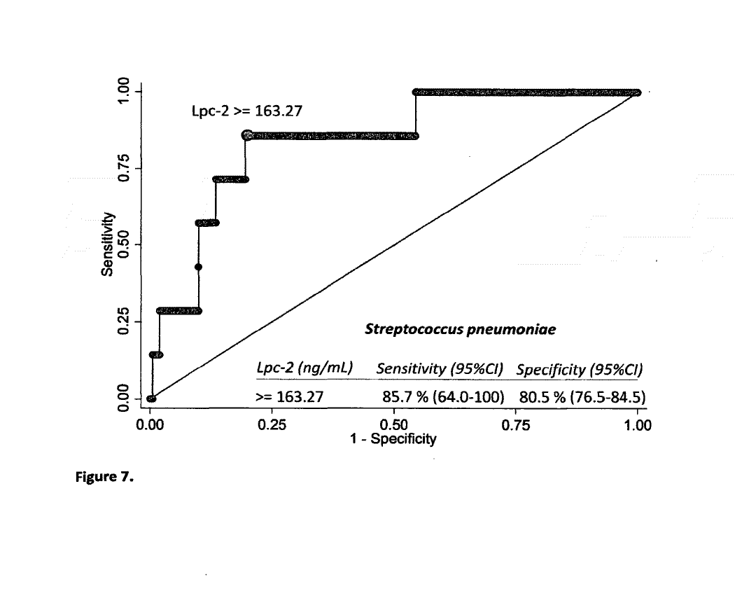

FIG. 7—ROC curve showing diagnostic performance of Lpc-2 to detect S. pneumoniae—positive blood cultures.

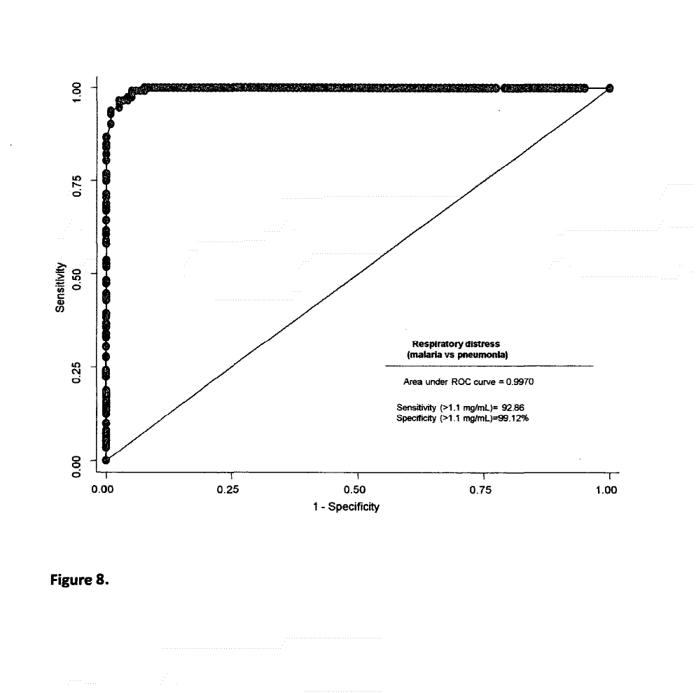

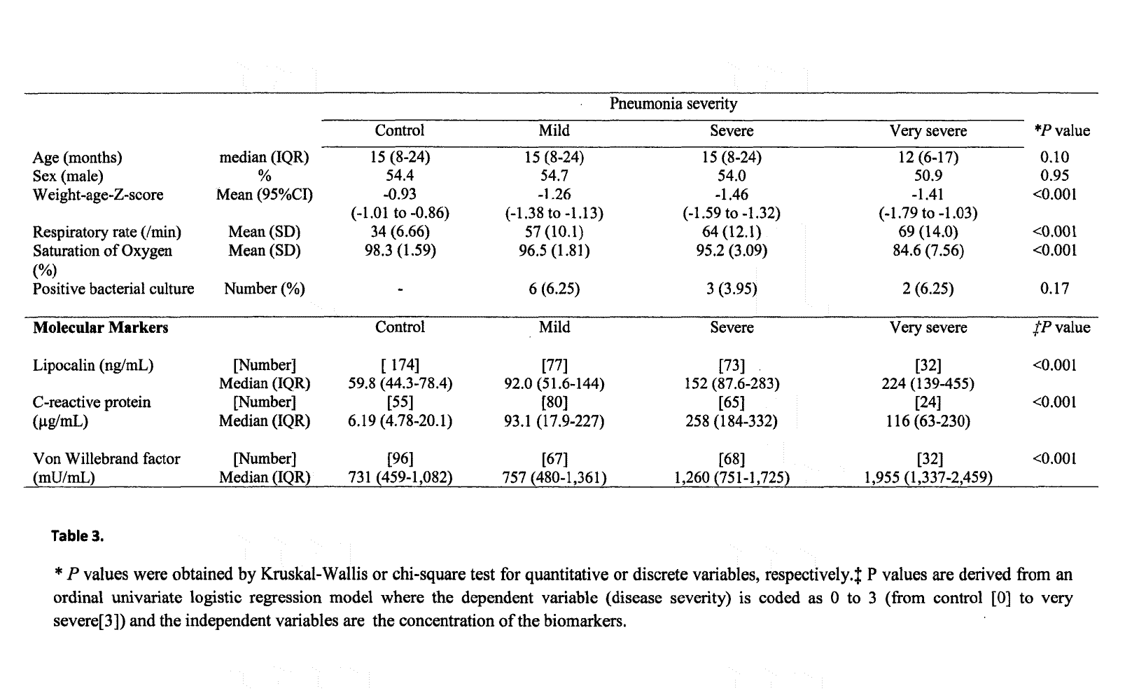

FIG. 8—shows ROC curve (sensitivity and specificity) showing the diagnostic performance of Haptoglobin (as a continuous variable) in Gambian children with respiratory distress. The outcome measure is severe malaria versus severe pneumonia, Haptoglobin identifies correctly those children with severe malaria or those with severe pneumonia.

### PATIENT STUDY—PNEUMONIA/PNEUMOCOCCAL INFECTION

### Samples

Plasma samples from 300 Gambian children aged 2 to 57 months were used for proteomic studies (see online methods for detailed methodology).

### Sample Treatment

### Plasma Depletion

Each sample was delipidated by centrifugation at 10,000×g for 10 min and clear plasma was collected in a new tube. The samples were diluted 8 times in buffer A (Agilent technologies, UK) and filtered through a 0.22 μm spin filter (Agilent technologies, UK) for 2 min at 16,000×g. For each run, 120 μL of diluted serum sample were injected into the Agilent Human 14 MARS Column (4.6×100 mm) coupled with 1200 Series HPLC (Agilent technologies, UK). Samples depletion was carried out using the following 48 min isocratic elution: 100% buffer A for 20 min at 0.125 mL/min then 2.5 min at 0.7 mL/min followed by 10 min of 100% buffer B (Agilent technologies) at 0.7 mL/min and 16.5 min of buffer A for the column equilibration. The flow-through was collected between 12 and 21 min with buffer A and the bound proteins were eluted between 26 and 30 min with buffer B. Each sample was injected several times in order to get sufficient proteins quantity for proteomics analysis.

### TCA/DOC Precipitation and Quantitation

The top 14 depleted samples were pooled and further concentrated and desalted by TCA/DOC precipitation. Sodium deoxycholate (final concentration: 125 μg/mL) was added to the samples and the mix left for 15 min at RT before the addition of trichloroacetic acid (final concentration: 6%). The samples were centrifuged for 10 min at 12,000 g. Ice cold acetone was added to the pellets and the samples were centrifuged at 10,000 g for 5 min at 4° C. The supernatants were discarded and the dried pellets were resuspended in 50 μL of buffer containing 6 M Urea and 100 mM Tris. The Pierce BCA protein assay (Thermo Scientific, Basingstoke, UK) was used for protein quantitation.

### SDS-PAGE and Protein Digestion

Equal amounts of the proteins isolated from the concentrated samples were separated onto a criterion XT Bis-Tris gel 4-12% using XT MES running buffer (Biorad, UK). After the separation of the proteins, the gels were stained with Instant Blue (Expedeon Ltd, Harston, UK) for 10 min and transferred in distilled water for direct use.

For each gel, fifteen bands were cut into small gel pieces (1-2 mm³), transferred into a 1.5 mL tube and destained with 50% Methanol and 5% acetic acid until colour disappeared. Each band was reduced with 10 mM DTT for 30 min, alkylated with 50 mM iodoacetamide for 30 min and digested with trypsin at 12.5 ng/mL in 50 mM ammonium bicarbonate at 37° C. overnight. Two incubations of 10 min with extraction buffer I (50% acetonitrile, 5% formic acid) and a third with extraction buffer II (85% acetonitrile, 5% formic acid) were used to extract the peptides from the gel pieces.

Peptide mixtures were dried with vacuum centrifugation and frozen at −20° C. until the MS/MS analysis.

### Mass Spectrometry Analysis

Samples were resuspended in 50 μl of Buffer A (2% acetonitrile, 0.1% formic acid) and subjected to LC-MS/MS (Waters, nAcquity, 75 μm×250 mm, 1.7 μm particle size) analysis using a Thermo LTQ Orbitrap Velos (Thermo Scientific, US) at a resolution of 30,000. MS/MS spectra were acquired in CID mode, selecting up to 20 precursors. Peptides were separated by a linear gradient of 1-40% Acetonitrile in 60 or 120 min at a flow rate of 250 nl/min.

MS/MS spectra were extracted from raw files by ProteomeWizard MSConvert (ref Bioinformatic application) using the 200 most intense peaks in each spectrum and converted into MGF-format peaklists. The peaklists were searched against the IPI human database (v.xyz, xyz entries) using Mascot (http://www.matrixscience.com/) v2.3.01, allowing one missed cleavage and 20 ppm/0.5 Da mass deviations in MS/MSMS. Carbamidomethylation of cysteine was a fixed modification. Oxidation of methionine and lysine and deamidation of asparagine and glutamine were used as variable modifications. For label-free protein quantitation Mascot results were imported into Progenesis LC-MS, Similar proteins were grouped and only non-conflicting features were used for quantitation.

### Database Searching and Label-Free Protein Quantitation

The identification of the proteins was done using mzXML files with the central proteomics facilities pipeline (Trudigan el al., Bioinformatics 36 (8):1131-1132 (2010)). The sequence database Human-Falc3D7, a concatenated database that includes both human and the Plasmodium falciparum 3D7 protein databases were analysed using the CPF Proteomics pipeline (Trudigan et al., Bioinformatics 36 (8):1131-1132 (2010)), which combines data from three search engines (Mascot, OMSSA and X!tandem k-score). The search was carried out using the following parameters. Trypsin was the enzyme used for the digestion of the proteins and only one missed cleavage was allowed. The accepted tolerance for the precursor was 50 ppm and 0.1 Da for the fragment. The search encompassed 1+, 2+and 3+ charge state, fixed modification for cysteine carbamidomethyl and variable modification for asparagine and glutamine deamidation, and methionine oxidation.

The label-free analysis was carried out using the normalised spectral index SINQ (Trudgian et al., Proteomics 11 (14):2790-2797 (2011)) and LC-MS Progenesis software (version 3.1.4003.30577).

Samples were separated into 3 different groups of 100 children according to disease severity (see FIG. 1). Individual samples (50 of plasma) were pooled into 3 different groups (˜165 μl of plasma per batch) in each disease category (control, non severe and severe pneumonia). Pooled plasma samples were depleted of top 14 highly-abundant plasma proteins (human serum albumin, IgG, haptoglobin, transferrin, IgA, alpha-1-antytrypsin, fibrinogen, alpha-2-macroglobulin, alpha-1-acid glycoprotein, complement C3, IgM, apolipoprotein A-I, A-II and transthyretin) with a multiple affinity removal (MARS) column (Agilent) using high-performance liquid-chromatography (HPLC). Proteins from depleted plasma were precipitated with trichloroacetone and quantified using a colorimetric assay (BCA Protein assay, Thermo Scientific, US) and further separated by size using SDS-PAGE (13 bands per sample). Protein bands were cut and digested with trypsin. Peptide digests were purified using Sep-Pak C18 columns (Waters, Milford, Mass.). Samples were analysed in an LC-MS/MS (LTQ Orbitrap Velos, Thermo Scientific, US) and searched against the human proteome with a false-discovery rate of 1% calculated from target-decoy hits and relative (label-free) quantification was based on normalised spectral counts (SINQ) (ref). Proteins that were up-or down-regulated following a disease progression pattern (SP>NSP>C or vice versa) in two batches or more were selected as candidate biomarkers. Final selection for enzyme-linked immunosorbent assays (ELISA) validation was based on these criteria and clinical and biological relevance. The concentration of selected proteins (C-reactive protein, von Willebrand factor and Lipocalin-2) was measured by ELISA (R&D Systems, UK) according to the manufacturer's instructions.

### Data Management and Statistical Analyses

Clinical data were collected on standardized paper forms, double entered into a database and verified against the original records. Univariate and multiple logistic regression models were fitted for all clinical variables to identify prognostic factors, using death as outcome variables using a significance level of 0.05. Goodness-of-fit was assessed by the Hosmer-Lemeshow test (P>0.05). In the multivariate analyses, the independent variables were checked for interaction. The area under the receiver operating characteristic (ROC) curves was used to compare the sensitivity and specificity of selected markers.

### Results

Proteomic Identification of Markers Associated with Severe Pneumonia

The plasma proteome of 200 Gambian children with pneumonia and 100 age- sex and location-matched controls was characterised using an unbiased shotgun protein strategy. A total of 23,212 peptides corresponding to 384 proteins were identified in 210 mass spectrometry runs of plasma samples from Gambian children with pneumonia and controls. 238 non-overlapping proteins were identified in children with severe pneumonia, 316 in children with non severe pneumonia and 268 in healthy age- and sex-matched controls. Relative abundance of proteins was quantified using label-free methods and selected for further validation if their relative abundance increased with disease severity (markers of disease progression) in two or more sample pools (see FIG. 2 and Table 1). 111 proteins were identified that were differentially regulated in two or more pools of samples. Of these, 20 were up-regulated in non-severe pneumonia cases compared to controls and 39 were up-regulated in severe compared to non-severe cases. Only 8 of 59 (13.5%) followed a disease progression trend from control to severe pneumonia: Lyzozyme (LYZ, IPI00019038) Lipocalin-2 (Lpc-2, IPI00299547), C-reactive protein (CRP, IPI00022389), von Willebrand factor (vWF, IPI00023014), serpin peptidase inhibitor (SERPINA3, IPI00550991), S100 calcium binding protein A8 (S100A8, IPI00007047), lipopolysaccharide binding protein (LBP, IPI00032311) and Leucine-rich alpha-2-glycoprotein 1 (LRG1, IPI00022417). Based on the magnitude of up-regulation across batches and biological and clinical meaning, CRP, Lpc-2 and vWF were selected for further validation.

### Diagnostic Performance of Clinical Features and Protein Biomarkers to Predict Severe Pneumonia

390 Gambian children were studied and univariate and multiple logistic regression analysis and ROC curves were used to evaluate the diagnostic performance of clinical features and molecular markers to predict severe pneumonia and very severe pneumonia. Clinical data were available for 96 non-severe pneumonia cases, 108 severe pneumonia cases (76 severe and 32 very severe pneumonia) and 186 age- sex and geographically-matched healthy controls. Lipocalin-2, CRP and vWF were measured in 356 patients, 224 patients and 263 patients, respectively.

To evaluate the clinical features associated with disease severity 96 children with mild pneumonia were compared with 108 children with severe pneumonia. Respiratory rate, presence of crepitations or crackles on auscultation, respiratory grunting, heart rate, low percentage oxygen saturation (<95%), inability to feed and pallor were associated with severe or very severe pneumonia (Table 2). Of these, respiratory rate was the best predictor of disease severity (AUC 0.76 [95% CI, 0.70-083]). The cut-off value with the highest sensitivity and specificity was 58 breaths per minute. Children with respiratory rates above 58 breaths per minute were 6.45 times more likely to have severe than non-severe pneumonia (OR 6.45 [95% CI, 3.49-11.9]). Respiratory rates greater than 58 breaths per minute predicted severe pneumonia with a sensitivity of 76% and a specificity of 61.4%. In the multivariable model, only respiratory rate (adjusted OR 3.9 [95% CI, 1.6-9.48]), crackles (adjusted OR 7.36 [95% CI, 2.57-21]) and inability to feed (adjusted OR 4.8 [95% CI, 1.9-12.4]) were independently associated with disease severity. The combination of the three clinical features had a sensitivity of 76.9% and a specificity of 72.2%.

Lipocalin-2, CRP and vWF were significantly higher in children with severe pneumonia compared with those with mild pneumonia as shown in FIG. 3. Lipocalin was the best predictor of severe pneumonia with a sensitivity of 72.3% and a specificity of 70.13% (AUC 0.71 [95% CI, 0.64-0.79). In children with Lpc-2 levels higher than 118 ng/mL, the risk of severity was nearly six-fold (OR 5.86 [95% CI, 3.07-11.1). CRP was associated with increased risk of disease severity with concentrations above 157 μg/mL (OR 2.94 [95% CI, 1.57-5.53]) but despite its good sensitivity to predict disease severity (70.8%), the specificity was low (56.2%), Similarly, vWF concentration higher than 648 mU/mL was associated with a five-fold increase in the odds of severe pneumonia (OR 5.26 [95% CI, 2.42-11.41]) but despite of high sensitivity (87%) the specificity was low (41.7%).

The combination of clinical and molecular markers that best predicted severe pneumonia included respiratory rate, crackles, Lpc-2 and CRP. This combination correctly classified 83.66% of children with severe pneumonia (FIG. 4). The sensitivity of this combination of markers increased to 94.7% (95% CI, 88.4-1.00) in children enrolled during the dry season (FIG. 5).

### Diagnostic Performance of Clinical Features and Protein Biomarkers to Predict Very Severe Pneumonia

To evaluate the clinical features associated with very severe disease, 73 children with severe pneumonia were compared with 35 children with severe pneumonia and oxygen saturation <90%. Respiratory rate, chest indrawing, pallor and bronchial breathing were significantly associated with very severe pneumonia in the univariate regression analysis. The odds of very severe disease were three times higher in children with a respiratory rate greater than 78 breaths per minute (OR 3.26 [95% C, 1.10-9.67]). In the multivariate analysis, respiratory rate, chest indrawing and pallor were independently associated with very severe pneumonia. The combination of these three clinical features was specific (80%) of very severe disease but sensitivity was low (54%). (FIG. 6)

Lpc-2, vWF and CRP were significantly associated with very severe pneumonia. Lpc-2 and vWF were significantly higher in patients with very severe pneumonia than in those children with severe disease, Children with severe pneumonia with Lpc-2 and vWF concentrations greater than 180 ng/mL and >1,915 mU/mL were 2.7 times and 5.2 times more likely to have very severe pneumonia, respectively. CRP concentration was significantly lower in very severe cases (FIG. 3). The diagnostic performance of the three molecules combined was specific (81.4%) but their sensitivity was low (66.6%).

The combination of clinical and molecular markers in the multivariable model included CRP, vWF, respiratory rate and pallor as variables independently associated with very severe pneumonia. The diagnostic performance of the combined model was not superior to the molecular or the clinical model separately.

### Discussion

A panel of molecular markers (Lpc-2, vWF and CRP) have been identified and validated that improve the diagnostic performance of clinical features to identify children with severe pneumonia (see description of population studied in Table 3). The combination of clinical features (respiratory rate and crepitations) and molecular markers (Lpc-2 and CRP) have a good sensitivity (84.8%) and specificity (82%) to predict severe pneumonia.

### PATIENT STUDY—MALARIA/MALARIAL INFECTION

Validation has been carried out in two independent studies that include a total of 1,224 patients with severe malaria, severe pneumonia and age-matched mild malaria, mild pneumonia and healthy controls. From a practical point of view, the results indicate that (1) respiratory distress in a child with a high plasma concentration of Lpc-2 (>112 ng/mL) is nearly 6 times more likely to be associated with severe disease (odds ratio 5.62, [95% CI 3.32-9.50]); and (2) haptoglobin correctly distinguishes 96.14% of children with respiratory distress and pneumonia from those with malaria (Sensitivity: 96.3% and Specificity: 95.8%). If developed into a diagnostic tool, these markers should have a positive clinical impact.

1. A method for determining the pneumococcal infection status of a subject, comprising the steps of:
i. providing a biological sample obtained from a subject;
ii. determining the level of lipocalin-2 in the biological sample; and
iii. comparing the level determined in (ii) with one or more pre-determined reference values.

2. A method for determining the pneumococcal infection status or malarial infection status of a subject, comprising the steps of:
i. providing a biological sample obtained from a subject;
ii. determining the level of lipocalin-2 and haptoglobin in the biological sample; and
iii. comparing the level determined in (ii) with one or more pre-determined reference values.

3. A method according to claim 2, wherein the method distinguishes severe pneumonia/pneumococcal infection from malarial infection.

4. A method according to any one of claims 1 to 3, wherein the biological sample is a plasma sample.

5. A method according to claim 1, wherein a level of lipocalin-2 greater than a concentration of 118 ng/mL is indicative of severe pneumonia or severe pneumococcal infection.

6. A method according to any one of claim 1 or 5, comprising determining the level of C-reactive protein and/or von Willebrand factor (vWF) in a biological sample from a subject and comparing the level of C-reactive protein and/or vWF with one or more pre-determined reference values.

7. A method according to claim 6, wherein a level of C-reactive protein greater than a concentration of 157 mg/mL is indicative of severe pneumonia or severe pneumococcal infection.

8. A method according to claim 6, wherein a level of vWF greater than a concentration of 1,915 mU/mL is indicative of severe pneumonia or severe pneumococcal infection.

9. A method according to claim 2, wherein a concentration of lipocalin-2 greater than about 98.64 ng/mL is indicative of acute respiratory distress and a concentration of haptoglobin greater than about 666,120 ng/mL is indicative of pneumonia as the cause of respiratory distress.

10. A method according to claim 2, wherein a concentration of lipocalin-2 greater than about 98.64 ng/mL is indicative of respiratory distress and a concentration of haptoglobin lower than about 627,000 ng/mL is indicative of malaria as the cause of respiratory distress.

11. A method for determining the appropriate treatment for a subject comprising the steps of:
(a) providing a biological sample obtained from a subject;
(b) determining the level of lipocalin-2 in the biological sample from said subject;
(c) comparing the level of lipocalin-2 determined in step (b) with one or more predetermined reference values; and
(d) using the results in (c) to determine the most appropriate therapy.

12. A method for determining the appropriate treatment for a subject comprising the steps of:
(a) providing a biological sample obtained from a subject;
(b) determining the level of lipocalin-2 and haptoglobin in the biological sample from said subject;
(c) comparing the level of lipocalin-2 and haptoglobin determined in step (b) with one or more predetermined reference values; and
(d) using the results in (c) to determine the most appropriate therapy.

13. A method of determining the severity of a pneumococcal infection or the response of a pneumococcal infection to a particular treatment in a subject comprising the steps of:
(a) providing a biological sample obtained from a subject;
(b) determining the level of lipocalin-2 in the biological sample from said subject; and
(c) comparing the level of lipocalin-2 determined in step (b) with one or more reference values.

14. A method of determining the severity of a malarial infection or pneumococcal infection or the response of a malarial infection or a pneumococcal infection to a particular treatment in a subject comprising the steps of:
(a) providing a biological sample obtained from a subject;
(b) determining the level of lipocalin-2 and haptoglobin in the biological sample from said subject; and
(c) comparing the level of lipocalin-2 and haptoglobin determined in step (b) with one or more reference value.

15. A kit for use in determining the diagnosis of severe/very severe pneumonia and/or severe/very severe pneumococcal infection in a subject comprising at least one agent for determining the level of lipocalin-2, in a biological sample provided by the subject.

16. A kit according to claim 15 comprising one or more agents for determining the level of one or more of C-reactive protein and/or vWF.

17. A kit for use in determining the diagnosis of severe malaria or severe pneumococcal infection/severe pneumonia in a subject comprising at least one agent for determining the level of lipocalin-2 and haptoglobin in a biological sample provided by the subject.

18. Use of the level of lipocalin-2, and optionally also one or more of C-reactive protein, vWF and/or haptoglobin, as a biomarker to determine the pneumococcal infection status of a subject.

19. Use of the level of lipocalin-2 and haptoglobin as a biomarker to distinguish between a pneumococcal infection and a malarial infection of a subject with respiratory distress.

20. Use of the level of lipocalin-2, C-reactive protein and/or vWF as a means of assessing the most appropriate therapy for an individual with a pneumococcal infection.

21. Use of the level of lipocalin-2 and haptoglobin as a means of assessing the most appropriate therapy for an individual with a pneumococcal infection or malarial infection.

22. A method for diagnosing pneumococcal infection in a patient comprising analysing a patient sample to determine the level of lipocalin-2, wherein pneumococcal infection is diagnosed if lipocalin-2 levels are elevated compared with one or more pre-determined reference values.

23. The method of claim 22 wherein if the level of lipocalin-2 in the sample is greater than about 118 ng/mL this is diagnostic of pneumococcal/pneumonia infection.

24. A method for diagnosing pneumococcal infection in a patient by analysing a patient sample to determine the level of lipocalin-2, and the level of one or more of C-reactive protein and von Willebrand factor (vWF), wherein pneumococcal infection is diagnosed if the level of lipocalin-2, and the level of one or more of C-reactive protein and von Willebrand factor is elevated compared with one or more pre-determined reference values.

25. The method of claim 24 wherein if the level of lipocalin-2 in the sample is greater than about 118 ng/mL, the level of C-reactive protein is greater than about 157 mg/mL and/or the level of vWF is greater than about 1,915 mU/mL this is diagnostic of pneumococcal infection/pneumonia.

26. A method for diagnosing and treating pneumococcal infection in a patient comprising: analysing a patient sample to determine the level of lipocalin-2, wherein pneumococcal infection is diagnosed if lipocalin-2 levels are elevated compared with one or more pre-determined reference values; and administering antibiotics or another therapy for pneumococcal infection, to a patient diagnosed with pneumococcal infection.

27. The method of claim 26 wherein if the level of lipocalin-2 in the sample is greater than about 118 ng/mL this is diagnostic of pneumococcal/pneumonia infection.

28. The method of claim 26 or 27 further comprising analysing a patient sample to determine the level of one or more of C-reactive protein and von Willebrand factor (vWF), wherein pneumococcal infection is diagnosed if the level of lipocalin-2, and the level of one or more of C-reactive protein and von Willebrand factor is elevated compared with one or more pre-determined reference values.

29. The method of claim 28 wherein if the level of lipocalin-2 in the sample is greater than about 118 ng/mL, and the level of C-reactive protein is greater than about 157 mg/mL and/or the level of vWF is greater than about 1,915 mU/mL this is diagnostic of pneumococcal infection/pneumonia.

30. A method for treating pneumococcal infection in a patient comprising: requesting a test to determine whether a patient has elevated levels of lipocalin-2 and administering antibiotics or another therapy for pneumococcal infection to a patient with levels of lipocalin-2 that are elevated compared to a reference value.

31. A method for diagnosing pneumococcal infection in a subject, wherein the pneumococcal infection is characterised by the presence of elevated levels of lipocalin-2 comprising:
i) providing a biological sample obtained from the subject;
ii) applying an antibody, preferably a monoclonal antibody, specific for lipocalin-2 to the sample, wherein presence of lipocalin-2 creates an antibody-lipocalin-2 complex;
iii) applying a detection agent that detects the antibody-lipocalin-2 complex; and
iv) diagnosing pneumococcal infection where an increase in the detection agents of step iii) is detected compared to a reference value.

32. The method of claim 31 wherein if the level of antibody-lipocalin-2 complex in the sample is greater than seen with a concentration of lipocalin-2 of about 118 ng/mL, then this is diagnostic of pneumococcal/pneumonia infection.

33. The method of claim 31 or 32 further comprising applying one or more antibodies specific for the C-reactive protein and/or von Willebrand factor (vWF).

34. A method for diagnosing malaria infection in a patient comprising analysing a patient sample to determine the level of lipocalin-2 and haptoglobin, wherein malaria infection is diagnosed if lipocalin-2 levels are greater than about 98.64 ng/mL and haptoglobin levels are lower than 627,000 ng/mL.

35. A method for diagnosing pneumococcal infection in a patient comprising analysing a patient sample to determine the level of lipocalin-2 and haptoglobin, wherein pneumococcal infection is diagnosed if lipocalin-2 levels are greater than about 98.64 ng/mL and haptoglobin levels are greater than 666,120 ng/mL.

36. A method substantially as herein described with reference to the examples and figures.

37. A kit substantially as herein described with reference to the examples and figures.

38. A use substantially as herein described with reference to the examples and figures.
